(19) United States
(12) Patent Application Publication
(10) Pub. No.: US 2026/0026932 A1
(43) Pub. Date: Jan. 29, 2026

(54) HEART VALVE MIMICRY

(71) Applicant: JenaValve Technology, Inc., Irvine, CA (US)

(72) Inventors: Payam SAFFARI, Aliso Viejo, CA (US); Vincent Ming Hai CHOW, Irvine, CA (US); Makensley LORDEUS, Costa Mesa, CA (US); Hussain S. RANGWALA, Villa Park, CA (US); Gregory Edwin MIRIGIAN, Tustin, CA (US); John F. MIGLIAZZA, Irvine, CA (US); Victoria E. CARR-BRENDEL, Irvine, CA (US)

(73) Assignee: JenaValve Technology, Inc., Irvine, CA (US)

(21) Appl. No.: 19/344,300

(22) Filed: Sep. 29, 2025

Related U.S. Application Data
(63) Continuation of application No. 17/643,988, filed on Dec. 13, 2021, now Pat. No. 12,433,745.
Application No. 17/643,988 is a continuation of application No. 16/481,287, now Pat. No. 11,197,754.
(60) Provisional application No. 62/451,296, filed on Jan. 27, 2017.

(51) Int. Cl. A61F 2/24; A61B 17/04

(52) U.S. Cl. CPC A61F 2/2418; A61B 17/0401; A61F 2/2415; A61F 2220/0008; A61F 2220/0016; A61F 2220/0075; A61F 2250/0025; A61F 2250/0039; A61F 2250/0097

(57) ABSTRACT

This disclosure relates to a heart valve with improved functionality and durability.

### CROSS-REFERENCE TO RELATED APPLICATIONS

This application is a continuation of U.S. patent application Ser. No. 17/643,988, filed on Dec. 13, 2021, now U.S. Pat. No. 12,433,745, which is a continuation of U.S. patent application Ser. No. 16/481,287, filed on Jul. 26, 2019, now U.S. Pat. No. 11,197,754, which is a U.S. national stage filing under 35 U.S.C. § 371 of International Application No. PCT/IB2018/050438, filed on Jan. 25, 2018, which claims the benefit of priority to U.S. Provisional Patent Application No. 62/451,296, filed on Jan. 27, 2017, the entire contents of each of which are incorporated herein by reference.

### DESCRIPTION

The present disclosure relates to a tent supported replacement heart valve useful in transcatheter treatment of a stenosis (narrowing) of a cardiac valve and/or a cardiac valve insufficiency.

The present disclosure also relates to an endoprosthesis (prosthesis) designed for anatomical positioning and mimicking the endogenous heart valve in a number of aspects exhibiting less interference and less side effects as known from prior art heart valves.

Specifically, the present disclosure also relates to a collapsible and expandable prosthesis incorporating a stent that can be delivered to the implant site using a catheter for treatment of a stenosis (narrowing) of a cardiac valve and/or a cardiac valve insufficiency.

The expression “narrowing (stenosis) of a cardiac valve and/or cardiac valve insufficiency” may include a functional defect of one or more cardiac valves, which is either genetic or has developed. A cardiac defect of this type might affect each of the four heart valves, although the valves in the left ventricle (aortic and mitral valves) are affected much more often than the right-sided part of the heart (pulmonary and tricuspid valves). The functional defect can result in narrowing (stenosis), inability to close (insufficiency) or a combination of the two (combined vitium). This disclosure relates to an endoprosthesis that includes an expandable stent capable of being implanted transluminally in a patient's body and enlarged radially or radially self-expanding after being introduced percutaneously or in any other known minimally invasive manner for treating such a heart valve defect.

In some treatments of severe narrowing of a cardiac valve and/or cardiac valve insufficiency, the narrowed or diseased cardiac valve is replaced with an endoprosthesis. Biological or mechanical valve models, which are typically surgically sewn into the cardiac valve bed through an opening in the chest after removal of the diseased cardiac valve, are used for this purpose. This operation necessitates the use of a heart-lung machine to maintain the patient's circulation during the procedure and cardiac arrest is induced during implantation of the prosthesis. This is a risky surgical procedure with associated dangers for the patient, as well as a long post-operative treatment and recovery phase. Such an operation can often not be considered with justifiable risk in the case of polypathic patients.

Minimally-invasive forms of treatment have been developed over the past decade which are characterized by allowing the procedure to be performed under local anesthesia. One approach provides for the use of a catheter system to implant a self-expandable stent to which is connected a collapsible valve that is, e.g., made from pericardial tissue. Such a self-expandable endoprosthesis can be guided via a catheter system to the implantation site within the heart through an inguinal artery or vein. After reaching the implantation site, the stent can then be released and allowed to self-expand.

A stent may be comprised of, for example, a plurality of self-expanding longitudinal stent segments, the segments being articulated relative to one another. In order to anchor the stent securely in position in an appropriate blood vessel close to the heart, anchoring barbs may be used to engage with the vascular wall.

An expandable stent for the fastening and anchoring of an endoprosthesis is known from publication DE 10 010 074 A1, whereby the stent is essentially formed from wire-shaped, interconnected segments. DE 10 010 074 A1 proposes a stent for fastening and anchoring an endoprosthesis, the stent having different arched elements which assume the function of fastening and supporting the valvular prosthesis at the site of implantation. Specifically, three identically-configured positioning arches spaced 120° from one another respectively are used. These positioning arches are connected to one another by means of solid body articulations. In addition to the positioning arches, complementary curved retaining arches serve to anchor the endoprosthesis by pressing radially against the vascular wall following the unfolding of the stent.

On the one hand, state of the art procedures and prostheses designs emphasize the convenience of the minimally invasive access and deployment procedure. On the other hand, known prosthesis design focuses on valve functions upon deployment, e.g., durability of the valve sutures to the stent. Other aspects relate to the Question of whether the prosthesis may remain in its position when the heart is pumping, e.g., that substantial forces act on the endoprosthesis during the filling phase of the heart cycle (diastole) and whether this can lead to endoprosthesis displacement longitudinally relative to the stent. Another focus of the prior art was the aspect of providing a secure seal to avoid peri-valvular leakage. Moreover, in the past there was awareness of positioning aspects like avoiding blockage of the coronary artery ostia (inlet orifice of coronaries) and the fact that such an interference of the prosthesis can lead to fatal coronary ischemia and myocardial infarction.

However, the prior art approaches and solutions focus on implantation procedures, basic valve functions and leakage aspects. Immediate and long term side effects of such a transcatheter heart replacement procedure and mimicking the endogenous heart valve are not at all addressed, or if mentioned, this aspect is not sufficiently addressed by the prior art.

Moreover, current trans aortic valve replacement therapy (TAVR) and known devices used in this procedure involve a number of disadvantages and risks like increased rate of stroke (about 4 to 6%) or the need for pacemaker implantation in treated patients depending on the device up to 25%. These issues are not sufficiently addressed or solved in known devices.

The prior art moreover does not appreciate the entire complexity of heart valve replacement therapy with regards to the functional implications, implant side effects such as pacemaker rate and the long term, post-procedural implications of such a treatment as well as with regards to sustainable long term effectiveness.

One problem of prior art approaches is that the prostheses do not sufficiently mimic the endogenous heart valve and accordingly, implantation of prior art prostheses results in an interference on a number of levels with the patients' heart and the endogenous heart function as well as a potentially disadvantageous impact on durability of the heart valve replacement therapy and unwanted long term side effects. Accordingly, the known transcatheter heart valve treatments do not represent an adequate and long-term solution for patients.

In view of the problems outlined above, one object of certain embodiments of the present application is to provide transcatheter heart valve prostheses with reduced side effects upon implantation in a patient, or at least to reduce or avoid the disadvantages of the prior art.

Another object of certain embodiments of the present application is to provide transcatheter heart valve prostheses exhibiting a reduced interference or substantially minimal interference with the endogenous functions of the heart of a patient, or at least to reduce or avoid the disadvantages of the prior art.

Another object of certain embodiments of the present application is to provide transcatheter heart valve prostheses which exhibit structural features which represent a long-term, durable transcatheter heart valve treatment of a patient, or at least to reduce or avoid the disadvantages of the prior art.

### SUMMARY OF THE DISCLOSURE

In one aspect, the disclosure relates to a heart valve replacement prosthesis which may have improved prosthesis alignment properties.

In another aspect, the disclosure relates to a heart valve replacement prosthesis comprising commissure and annulus alignment means.

In another aspect, the disclosure relates to a heart valve replacement prosthesis comprising one or more of the following characteristics which, alone or in combination, may provide for better of mimicking the endogenous heart valve and may lead to less interference with the patient's heart:
1—Number of cells per cm² area is 2 to 6, and more preferably 3.7 to 5.1 in the inflow end region and 1 to 4, and more preferably 1.9 to 2.5 in outflow end region, or/and
2—In the inflow end region the ratio of area of any given cell to a neighboring cell is 0.25 to 4, and more preferably 1, or/and
3—In the inflow end region the ratio of height of any given cell to a neighboring cell is 0.5 to 2, and more preferably 1, or/and
4—In the inflow end region the aspect ratio of all cells is between 1 to 2, more preferably 1.5 to 1.6. Aspect ratio may be defined as a ratio of height to width of cells, or/and
5—In the outflow end region, aspect ratio of cells varies from 1 to 3, and more preferably 1.1 to 2.9. Aspect ratio may be defined as a ratio of height to width of cells, or/and
6—In the outflow end region, the cells vary in area in a single circumferential row and the area of adjacent cells in circumferential rows varies from 5 to 250 mm², and more preferably 7 to 198 mm², or/and
7—In the outflow end region, the cell heights vary in a single circumferential row and the height of adjacent cells in circumferential rows varies from 3 to 30 mm, and more preferably 6 mm to 25 mm.

In another aspect, the disclosure relates to a heart valve replacement prosthesis that may exhibit advantageous hemodynamics and mimic or essentially mimic the natural hemodynamics of native valve function.

In another aspect, the disclosure relates to a method of implantation of a replacement heart valve prosthesis.

In another aspect, the disclosure relates to a method of treating a diseased or stenotic heart valve or otherwise dysfunctional heart valve.

### BRIEF DESCRIPTION OF THE FIGURES

FIG. 1 describes the annulus diameter (11) and the annulus-to-commissure distance (12) which are relevant in certain aspects of the disclosure.

FIG. 2 describes an endogenous heart valve with the annulus diameter (AD) and annulus-to-commissure distance (ACD) relationship to an exemplary embodiment of the disclosure which may exhibit advantageous alignment and mimicry of the natural heart valve.

FIG. 3 describes a top view and a side view of an exemplary prosthesis with commissure alignment means which are positioned at the proximal end.

FIG. 4 describes another exemplary prosthesis with commissure alignment means which are positioned at the proximal end and represent a variation of the design in FIG. 3.

FIG. 5 describes an exemplary prosthesis of the disclosure placed in the endogenous heart valve, depicting the hemodynamic flow which may essentially mimic the natural hemodynamics of a fully functional endogenous heart valve.

### DETAILED DESCRIPTION OF THE DISCLOSURE

As used herein, the terms “comprises,” “comprising,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” as used herein is used in the sense of “example,” rather than “ideal.”

As used herein, the terms “about,” “substantially,” and “approximately,” may indicate a range of values within +/−5% of a stated value.

At least some problems of the prior art may be solved by a replacement heart valve prosthesis comprising a stent component and a valve component characterized by a valve ratio of annulus to commissure distance to annulus diameter of about 1.01 to 1.14 and preferably of about 1.03 to 1.04, or/and the number of cells per cm² area is about 2 to 6 and more preferably 3.7 to 5.1 in the inflow end region and about 1 to 4 and more preferably 1.9 to 2.5 in the outflow end region, or/and in the inflow end region the ratio of area of any given cell to a neighboring cell is about 0.25 to 4 and more preferably about 1, or/and in the inflow end region the ratio of height of any given cell to a neighboring cell is 0.5 to 2 and more preferably 1, or/and wherein the inflow end region the aspect ratio of all cells is about 1 to 2 and more preferably about 1.5 to 1.6, or/and wherein the outflow end region the aspect ratio of cells varies from about 1 to 3, preferably about 1.1 to 2.9 or/and wherein in the outflow end region the cells vary in area in a single circumferential row and the area of adjacent cells in circumferential rows varies from about 5 to 250 mm², preferably about 7 to 198 mm² or/and wherein the outflow end region, the cell heights vary in a single circumferential row and the height of adjacent cells in circumferential rows varies from about 3 mm to 30 mm and more preferably from about 6 mm to 25 mm, or/and a radial force distribution of the prosthesis and the stent where about 15 to 25% of the radial force is provided by the annulus region and about 75 to 85% of the radial force provided by the supra-annulus region or/and the distance of the proximal end of the commissure alignment means to the distal end of the inflow end section is 4 to 12 mm, preferably 7.5 to 9 mm.

One advantage of certain embodiments of the disclosure is that they provide a means to align the commissures of the prosthesis with the native valve commissures. This advantage may be achieved by positioning means that protrude into the native valve pockets or by positioning means that achieve the alignment by way of self-aligning to the native commissures. Accordingly, the aligning means are attached or are a part of the prosthesis as appropriate for the respective purpose, e.g., to be positioned in the native valve pockets or at the native commissures, and thus these means protrude some distance or length from the main body of the prosthesis or they may represent an aligning means of the prosthesis to fit directly to the native commissures.

Another advantage of certain embodiments of the disclosure is that the stent is designed to reduce or essentially entirely avoid the risk of stenosis. For patients with aortic stenosis, the supra-annulus region, particularly the valve leaflets, is the most diseased area and it has been shown in TAVR procedures that stenosis occurs primarily within and around this region. At least one embodiment of the disclosure provides one solution in this respect by way of its specific design. The stent that supports the pericardial valve of the prosthesis may have differentiating chronic outward force (CoF) along the length of the THV. It may provide essentially the most outward force in the diseased area, e.g., supra-annulus region, and lower outward force in the annulus region of the native vessel. This may be useful because stenosis occurs primarily in the valve leaflets in the supra-annulus region and not in the annulus region.

The design and geometry of the stent described in this disclosure may achieve a radial force distribution of about 15 to 25% of the radial force provided by the annulus region and about 75 to 85% of the radial force provided by the supra-annulus region.

Another advantage of certain embodiments of the disclosure is that the specific design of the prosthesis may provide an anatomical alignment beyond pure commissure alignment. Hence, the structural alignment of the prosthesis with the native valve described in certain embodiments of the disclosure may preserve flow dynamics similar or essentially similar to the endogenous native valve with fewer side effects in comparison to prior art replacement heart valves.

The advantages of certain embodiments of the disclosure may be achieved in one aspect by the strut design, strut dimensions and strut distribution.

The advantages of certain embodiments of the disclosure are also achieved in one aspect by way of a stent cell density and stent cell distribution in different sections of the stent. The inflow end region at the distal end of the stent is generally located below the annulus in the ventricle and the outflow end region is generally located at the proximal end of the stent above the annulus. Both regions exhibit a specific stent cell or/and material distribution which may contribute to the advantageous functional aspects of the disclosure.

Another advantage is that the prosthesis provides in one aspect for “circumferential alignment” of the prosthesis valve commissures and replacement valve essentially with the endogenous commissures and valve leaflets. This may preserve natural valve functionality with respect to circumferential alignment and may be relevant for minimizing or eliminating unwanted side effects due to any potential change in flow pattern. “Circumferential alignment” with regard to the disclosure may relate to the alignment of the prosthesis commissures to the natural commissures. “Axial alignment” may relate to the alignment of the vertical level of the replacement valve with the endogenous valve. One may achieve essential circumferential and axial alignment of both replacement commissures and valve with their endogenous counterparts. Accordingly, such structural alignment may achieve flow dynamics and pressure distribution after implantation of the replacement heart valve prosthesis and during heart function that resembles and essentially mimics natural heart valve function.

Another advantage is that the prosthesis described in this disclosure may replicate the hemodynamic properties of the native aortic valve. Circumferential alignment of the commissures combined with alignment of the height of the prosthetic leaflets with the height of the native leaflets results in the prosthetic valve opening and closing in a similar manner and position as the native valve, thus may preserve the natural flow dynamics of the native valve. Also, the prosthesis may not block the sinuses of the aortic root with the stent frame or the tissue which allows blood to freely flow in areas when the valve is closed and may not interfere with the natural formation of eddy currents in the region.

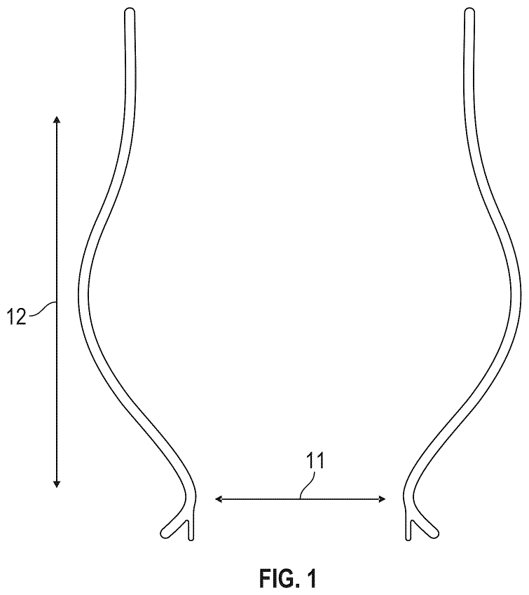

FIG. 1 describes the annulus diameter dimension (horizontal arrows) and the endogenous annulus to upper commissure edge (proximal endogenous commissure edge) distance (vertical arrows). The disclosure achieves an alignment and relative positioning to mimic the endogenous dimensions without extending the prosthesis into heart areas such as the ascending aorta or left ventricle. Advantageously the prosthesis may avoid interference with endogenous functional areas or areas of functional significance, and also may avoid issues or complications that arise from TAVR procedures such as perivalvular leak and the need for permanent pacemaker implantation.

The term “aortic valve insufficiency”, or “aortic insufficiency” for short, may refer to the defective closing of the heart's aortic valve and the diastolic reverse flow of blood from the aorta into the left ventricle as a result. Depending on the severity of the aortic insufficiency and the extent of resistance to aortic depletion, the volume of reverse flow can be up to two thirds of the left ventricle's ejection volume (normal cardiac output 40 to 70 ml). This results in a characteristically high blood pressure amplitude. This regurgitated blood flow increases the diastolic filling of the left chamber and leads to a volume overload of this section of the heart, a consequence of which is eccentric hypertrophy.

Structural features which contribute to some embodiments of the disclosure and which may advantageously contribute are the alignment of the THV prosthesis leaflets with the geometry of the endogenous valve leaflets, and/or the alignment of the THV commissures with the endogenous valve commissures and/or the THV stent cell seize over the length of the THV, e.g. a higher density of cells in the annulus region and a lower density of cells in the outflow region.

In another aspect, structural features which contribute to the advantageous results of the prosthesis are commissure alignment means, a tissue fastening portion and the prosthesis alignment with the endogenous leaflet geometry and the distal distance of the commissure alignment means to the distal end of the THV.

In preferred embodiments, the disclosure relates to a prosthesis as described above wherein the stent further comprises at least one alignment means, preferably two, three, four, five or six alignment means.

In another aspect, the prosthesis of the disclosure is characterized in that the alignment means is a feeler to be introduced into the native cusp(s) or/and a commissure alignment means to engage with the native valve commissure(s) and which is part of or fixed to the proximal end of the prosthesis.

In another aspect, the prosthesis of the disclosure is characterized in that the stent further comprises a fastening portion for fixation of the valve tissue, preferably pericardial tissue. Preferably the fastening portion is an arch, preferably comprising holes or rails or notches.

In another aspect, the disclosure relates to the use of the prosthesis as described herein in the treatment of heart valve stenosis or heart valve insufficiency.

In another aspect, the disclosure relates to a method for treating heart valve stenosis or heart valve insufficiency comprising the steps of loading or using a preloaded prosthesis as described herein with a delivery catheter useful for vascular deployment, introducing the catheter in a patient's vasculature, protruding and positioning the catheter for deploying the prosthesis at the target site, releasing and deploying the prosthesis at the target site and retracting the catheter from the patient's vasculature.

Of particular advantage is the specific geometry of the prosthesis according to some embodiments of the disclosure, which may result in fewer side effects after implantation as compared with known replacement heart valves.

The prosthesis may be characterized by the following advantageous features with regards to stent and prosthesis design which may lead to superior long term functionality and/or less side effects. The prosthesis described in this disclosure may exhibit one of several of the following features in combination.

In one aspect the “dimensions” and relative proportions of stent features and sections to each other—and thus the prosthesis including valve support—are relevant to mimic and achieve desired prosthesis function in the patient and to reduce or essentially avoid unwanted side effects.

One can divide/define the prosthesis according to the disclosure in various sections. In one aspect, the prosthesis can be structured axially where the prosthesis has an inflow end region at a distal end of the frame section and an outflow region at a proximal end of the frame.

According to literature, the ratio of annulus to commissure distance (ACD) to annulus diameter (AD) is typically 0.85-1.14 with an average of 1.04 for patients (H. Ruel et. al. 1990).

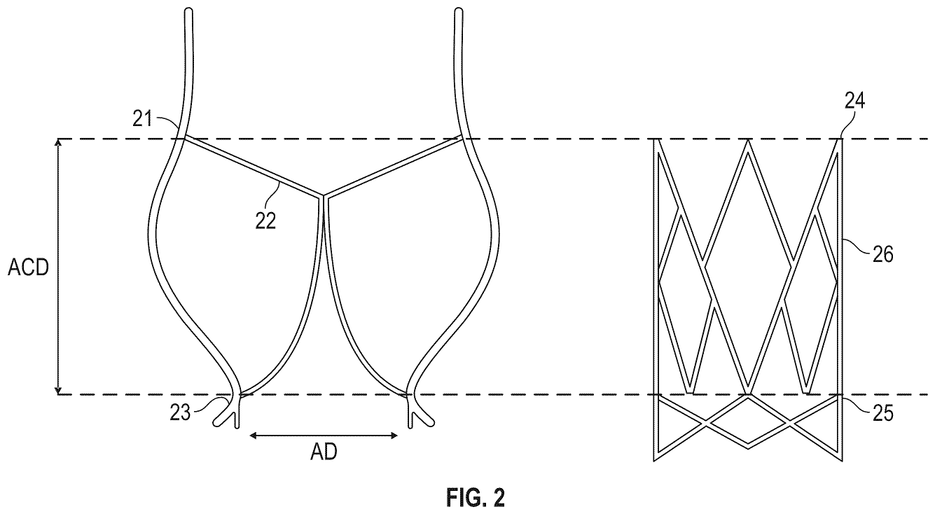

The design and geometry of the prosthesis described in this disclosure may achieve a valve ratio of annulus to commissure distance to annulus diameter of about 1.01 to 1.14 and preferably 1.03 to 1.04 depending on the annulus diameter of the native valve. Details are also depicted in FIG. 2.

Another aspect is that the annular geometry of the stent is independent from the geometry of the proximal section. In endogenous valve geometry, the annulus is generally non-circular and the commissure region is circular. In its implanted state, the stent of the prosthesis in the inflow end region maintains non-circular geometry while the proximal end of the outflow region maintains circular geometry. The stent achieves this by way of the height of the outflow end region, or the aforementioned ACD (annulus to commissure distance) to AD (annulus diameter) ratio.

In another aspect, one can also structure the prosthesis horizontally, e.g., in two sections which may be repeated two or three times.

Another aspect is the “circumferential stent cell distribution” or “circumferential area coverage of adjacent cells” in general or within regard to the different sections as described above.

Cell distribution according to the disclosure can be defined by various parameters and characteristics. Cell distribution features which create advantageous structural and functional performance characteristics after implantation, depending on the diameter of the native valve, include any one or more of the following:

Cell density, or the number of cells per cm² area can be about 2 to 6, preferably 3.7 to 5.1 in the inflow end region and about 1 to 4, preferably 1.9 to 2.5 in the outflow end region.

In the inflow end region, the ratio of area of any given cell to the neighboring cell is about 0.25 to 4, and more preferably about 1.

In the inflow end region, the ratio of height of any given cell to the neighboring cell is 0.5 to 2, and more preferably 1.

In the inflow end region, the aspect ratio of all cells is between 1 to 2, preferably 1.5 to 1.6. Aspect ratio may be defined as the ratio of cell height to cell width.

In the outflow end region, the aspect ratio of cells varies from about 1 to 3, and more preferably about 1.1 to 2.9. Aspect ratio may be defined as ratio of cell height to cell width.

In the outflow end region, the area of adjacent cells within circumferential rows varies from about 5 to 250 mm², and preferably about 7 to 198 mm².

In the outflow end region, the height of adjacent cells in circumferential row varies from about 3 to 30 mm, preferably from about 6 mm to 25 mm.

The stent (THV or prosthesis frame or support frame) of the prosthesis according to the disclosure may exert radial force on the aortic annulus and aortic root wall after deployment. This radial force may be primarily responsible for maintaining the prosthesis in the correct position. Frictional force between the aortic root/annulus and the stent may result from the radial force of the stent while the feelers create mechanical interference between each feeler tip and native aortic cusp.

Another aspect of the disclosure is the distance of the proximal end of the commissure alignment means to the distal end of the inflow end section, which is important for ensuring that the final height and position of the inflow end region is at a proper height in the annulus so as to prevent the need for permanent pacemaker implantation. In one particular embodiment of this disclosure, this distance is 4 to 12 mm, and most preferably 7.5 to 9 mm.

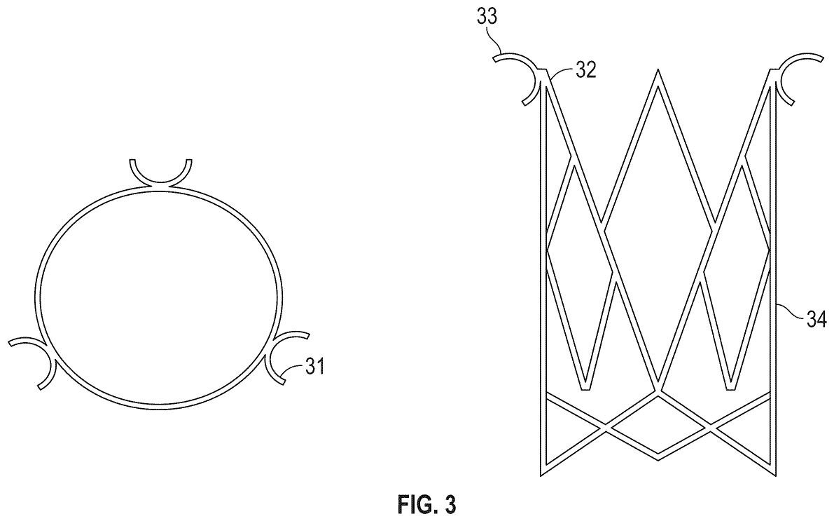

Another aspect is a commissure alignment means which is positioned at the proximal end of the prosthesis, which can be one, two or three means. It is formed to allow for engagement with the endogenous commissures of the valve and to prevent the prosthesis from axial movement in the distal direction (toward the ventricle) and to align the prosthesis with the endogenous commissures, thus mimicking the endogenous anatomical alignment. In combination with previously disclosed dimensional features of the prosthesis and the stent, this proximal commissure alignment means may also allow for vertical positioning of the distal inflow end region of the valve according to the position of the endogenous valve. This goal can alternatively be achieved by an alignment means (one, two or three) which is attached to or forms an integral part of the stent in combination with the previously disclosed dimensional features of the stent which are adapted accordingly. In FIG. 3 an exemplary embodiment is depicted.

The endogenous positioning of this disclosure can be achieved by way of using visual markers affixed to the prosthesis and monitoring them during the deployment procedure for correct positioning of the prosthesis. Of particular usefulness to some embodiments according to the disclosure can be alignment means as described above and below.

As described herein, stents may be radially expandable intravascular implants capable of being implanted vascularly, preferably transfemorally, and enlarged or self-expanding radially after being introduced percutaneously. The stents may be configured to be placed in a native diseased valve of a patient, such as a native stenotic aortic or pulmonary valve, using a minimally-invasive approach, such as a beating heart transapical procedure or a retrograde transaortic procedure. Although stents can be introduced into the body of the patient via any number of access points, a transvascular approach by femoral access or by transapical access for the aortic valve is preferred. However, this disclosure is not limited to these approaches.

A “native aortic valve” may be a valve with three leaflets or congenitally bicuspid with two leaflets.

An endoprosthesis may include an implant which (together with a stent to which the valvular prosthesis is affixed) functions as a check valve, opening to permit forward blood flow and closing to prevent retrograde flow. A valvular prosthesis may consist of at least two, and preferably of three leaflets, and a valve skirt on which the leaflets are connected.

At least one fastening portion extends along the longitudinal axis of the stent and comprises a plurality of fastening holes distributed in a longitudinal direction at discrete positions along the length of the at least one fastening portion. Thread or thin wire may be guided through each fastening hole to secure the valvular prosthesis to the stent. One advantage of this feature may be that longitudinal displacement of the valves relative to the stent is substantially minimized once implanted and so the prosthesis is not unduly disturbed or weakened as a result of the heart's peristaltic motion.

In addition, the stent may contain fastening means comprised of holes, and/or may include one or more notches to assist the seating and retaining of suture material. The notches also may assist with even attachment of the prosthesis to the stent and, similarly to the fastening holes, minimize longitudinal displacement of the prosthesis.

Extending from and between a pair of fastening portions is a fastening arch, to which valve tissue is attached or over which valve tissue is laid. In the expanded and implanted state of the stent and the valvular prosthesis affixed thereto, the fastening arch of the stent abuts against the vessel wall at least at the lower section of the stent in order to seal against leakage. Furthermore, with the fastening arch, the prosthesis tissue is separated and held away from other parts of the stent, thereby reducing the likelihood of these stent parts chaffing the tissue which, in turn may result in damage and weakening of the prosthesis.

The stent may comprise alignment means being an integral part of the stent or affixed thereto at the appropriate position of the stent which aide in alignment of the valve of the prosthesis with the natural commissures. Such means may be at least one feeler, or possible two or three feelers being positioned in the cusp(s) of the natural aortic valve. Such an alignment means may also be a commissure alignment means allowing for commissure alignment. This can be achieved by way of aligning the commissure alignment means themselves proximal to the endogenous commissures, and/or engaging with the proximal part of the endogenous commissures and thus aligning the prosthesis commissures with the endogenous commissures. Thus, the prosthesis' commissures may be positioned in alignment with the natural commissures in the patient's heart.

In an implanted configuration, at least one aligning means of the stent extends from the circumference of the stent in a generally radial direction.

In manufacturing the stent used in the valvular prosthesis according to a particular embodiment of the disclosure, the stent may exhibit a structure integrally cut from a portion of tube, in particular from a metal tube, which incorporates all components of the stent. It is also conceivable that the stent is cut out of a relatively large tube, e.g., a tube having a diameter which is larger compared with the diameter of the final stent in its collapsed configuration. For example, a tube having a diameter of approximately 10 mm may be used for cutting a specific stent pattern into this tube. Then the cut pattern may be different, as it may become necessary to crimp the stent to something smaller than what it was originally cut from. In particular, with this procedure it is possible to remove material during cutting and processing in a defined manner thereby enhancing the functionality of the final stent.

The stent preferably exhibits an integrally-formed structure which can transform from a first predefinable shape into a second predefinable shape, whereby the stent exhibits a first predefinable shape (collapsed shape) during insertion into the patient's body and a second predefinable shape (expanded shape) once implanted.

In a preferred embodiment, the stent exhibits in its first shape (collapsed shape) an outer diameter of approximately 4 to 8 mm and a length of between 30 mm and 48 mm. More precisely, the stent may exhibit in its first shape (collapsed shape) an outer diameter of approximately 4.0 to 8.0 mm, preferably 6.0 to 6.7 mm, and a length of between 33.0 mm and 45.0 mm, and preferably between 36.0 mm and 42.0 mm. This allows a prosthesis including the stent to be inserted easily into the patient's body, for example with a 19 F or 21 F delivery system, and to be used with an endoprosthesis having a diameter of between 19 mm and 28 mm. The afore-mentioned length specifications are the dimensions currently preferred, based on which the stent becomes suitable for the majority of patients to be treated.

In a particularly preferred realization, the stent comprises a valvular prosthesis, preferably a biological or pericardial valvular prosthesis, wherein the tissue component(s) of the valvular prosthesis is/are attached to the at least one fastening portion of the stent by means of a thread or the like. A commissure alignment means form also part of a preferred prosthesis, and in a more preferred embodiment the alignment means may lead to commissure and annulus alignment.

In one realization the stent and thus the prosthesis exhibits an advantageous radial compliance. The three sections of the stent as defined above may have a varying elasticity from section 1 to 2 to 3, or from section 1 to 2, respectively. The inflow end region (section 1) may exhibit a relative low elasticity and a relatively high stiffness. Section 2 (when dividing the prosthesis in two sections) may exhibit a higher elasticity and less stiffness. In case the stent is divided into three sections there exists a gradient from section 1 to section 2 to section 3.

Moreover, the advantageous stent design may allow for mimicking the endogenous valve behavior when the heart is pumping. One design feature and advantage of this aspect of the disclosure is that the annulus area of the prosthesis (or inflow end region) may essentially maintain its diameter. The proximal section (section 2 or sections 2 and 3, respectively) may allow movement due to their higher elasticity. Thus the commissures of the valve and the whole diameter of the stent (and thus of the prosthesis) are capable to move inwardly when the valve closes and the diameter of section 3 (or section 2 respectively) may be reduced and thus mimic the endogenous and native valve. Thus the inflow end of the prosthesis when implanted may resemble natural, endogenous functionality wherein the diameter remains essentially constant and the proximal prosthesis section varies its diameter and reduces its diameter when the valve closes. The diameter of the proximal end can reduce by 1 to 3 mm, or 1 to 2 mm.

A shape memory material is preferably used as the material for the stent, the material being designed such that the stent can transform from a temporary shape into a permanent shape under the influence of an external stimulus. The temporary shape is thereby the stent's first shape (e.g., the collapsed state of the stent), while the permanent shape is assumed in the stent's second shape (e.g., in the expanded state of the stent). In particular, use of a shape memory material such as Nitinol, e.g., an equiatomic alloy of nickel and titanium, allows for a particularly gentle implantation procedure when implanting the stent.

When manufacturing the stent preferably is made from a shape memory material, the stent structure is preferably shaped after it has been cut from a tube. It is conceivable that the stent is cut out of a tube having a diameter which is larger compared with the diameter of the final stent in its collapsed configuration. Then, the laser-processed tube is crimped thereby achieving the diameter of the stent in its collapsed configuration. Once the desired shape has been formed, this shape is “fixed”, this process being known as “programming”. Programming may be effected by heating the stent structure, forming the stent into the desired shape and then cooling the stent. Programming may also be effected by forming and shaping the stent structure at lower temperature, this being known as “cold stretching.” The permanent shape is thus saved, enabling the stent to be stored and implanted in a temporary, non-formed shape. If an external stimulus then acts on the stent structure, the shape memory effect is activated and the saved, permanent shape restored.

A particularly preferred embodiment provides for the external stimulus to be a definable transformation temperature. It is thus conceivable that the stent material needs to be heated to a higher temperature than the transformation temperature in order to activate the shape memory effect and thus recover the stored, permanent shape of the stent. A specific transformation temperature can be preset by the relevant selection of the chemical composition of the shape memory material.

It is particularly preferred to set the transformation temperature to be in a range between 10° C. and the patient's body temperature, and preferably in the range of between 10° C. and room temperature. Doing so may be advantageous, especially with regards to the medical device being used as an implant in a patient's body. Accordingly, all that needs to be ensured in this regard when implanting the stent is that the stent is warmed up to room temperature or the patient's body temperature (37° C.) at the site of implantation to activate the shape memory effect of the stent material.

The present disclosure as described above relates to a heart valve prosthesis (endoprosthesis) comprising an expandable or self expanding stent and a valve used in the treatment of a stenosis (narrowing) of a cardiac valve and/or a cardiac valve insufficiency. Furthermore, the present disclosure relates to a collapsible and expandable prosthesis incorporating a stent that can be delivered to the implant site using a catheter for treatment of a stenosis (narrowing) of a cardiac valve and/or a cardiac valve insufficiency. Although the stent and the valvular prosthesis affixed thereto can be used for replacing any of the four different heart valves, in particular the pulmonary valve and the aortic valve, the application of the disclosure for treatment of a diseased aortic valve has been described above only for reasons of simplification.

The above disclosure is intended to be illustrative and not exhaustive. This description may suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the disclosure such that the disclosure should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g., each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

### REFERENCE NUMBER LIST

### FIG. 1:

11 Annulus diameter (AD)
12 annulus-to-commissure distance (ACD)

### FIG. 2:

21 Native commissure
22 Native leaflet
23 Native annulus
24 THV commissure
25 THV inflow end section
26 THV

### FIG. 3:

31 Commissure alignment mean
32 THV commissure
33 Commissure alignment mean
34 THV

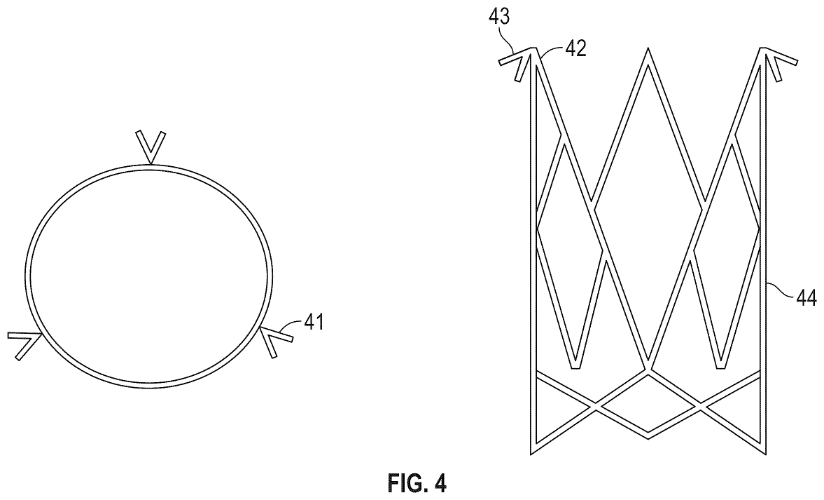

### FIG. 4:

41 Commissure alignment mean
42 THV commissure
43 Commissure alignment mean
44 THV

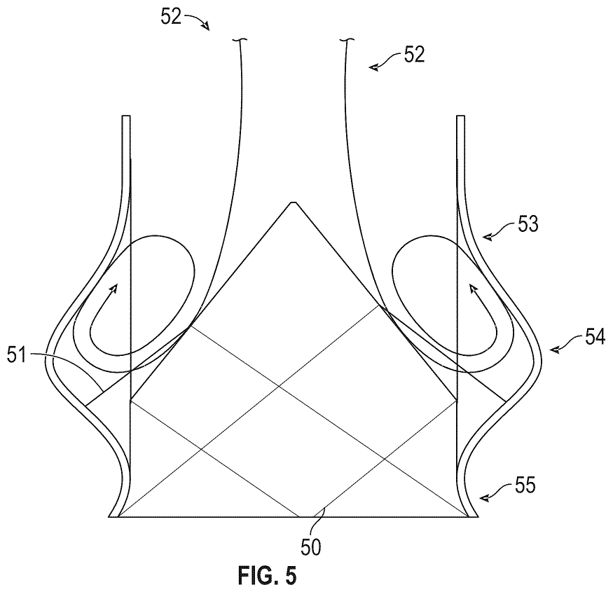

### FIG. 5:

50 Valve
51 Alignment means
52 Flow
53 STJ
54 Sinus
55 Annulus

What is claimed is:

1. A replacement heart valve prosthesis configured to be implanted at a native heart valve, the replacement heart valve prosthesis comprising:
a valve component having a valve ratio of annulus to commissure distance (ACD) to annulus diameter (AD) of 1.01 to 1.14, the ACD being a distance between a proximal end of a commissure portion and a proximal end of an annulus portion; and
a stent component comprising a plurality of barbs, an inflow end region and an outflow end region each comprising a plurality of cells, wherein in the inflow end region, a ratio of area of a given cell to a neighboring cell is 0.25 to 4, a ratio of height of the given cell to the neighboring cell is 0.5 to 2, or an aspect ratio of all cells of the inflow end region is 1 to 2, and wherein in the outflow end region, aspect ratios of a first cell and a second cell in a single circumferential row vary with respect to one another from 1 to 3, areas of the first cell and the second cell vary with respect to one another from 5 mm² to 250 mm², or heights of the first cell and the second cell vary with respect to one another from 3 mm to 30 mm,
wherein the plurality of barbs are configured to engage with a vascular wall for anchoring the replacement heart valve prosthesis.

2. The replacement heart valve prosthesis of claim 1, wherein the stent component includes at least one feeler for aligning the stent component relative to a native heart valve.

3. The replacement heart valve prosthesis of claim 2, wherein the at least one feeler is configured to be introduced into a native cusp of the native heart valve.

4. The replacement heart valve prosthesis of claim 2, wherein the stent component includes three feelers for aligning the stent component relative to native cusps of the native heart valve.

5. The replacement heart valve prosthesis of claim 2, wherein the stent component includes exactly three feelers for aligning the stent component relative to the native heart valve.

6. The replacement heart valve prosthesis of claim 1, wherein the valve component comprises tissue, and the stent component further comprises an arch for fixation of the tissue.

7. The replacement heart valve prosthesis of claim 6, wherein the tissue comprises pericardial tissue.

8. The replacement heart valve prosthesis of claim 6, wherein the arch comprises holes, rails, or notches.

9. The replacement heart valve prosthesis of claim 1, wherein the stent component comprises a ring of cells in the inflow end region.

10. The replacement heart valve prosthesis of claim 1, wherein the stent component is self-expanding.

11. A replacement heart valve prosthesis configured to be implanted at a native heart valve, the replacement heart valve prosthesis comprising:
a valve component having a valve ratio of annulus to commissure distance (ACD) to annulus diameter (AD) of 1.01 to 1.14, the ACD being a distance between a proximal end of a commissure portion and a proximal end of an annulus portion; and
a stent component comprising an inflow end region and an outflow end region each comprising a plurality of cells, wherein in the inflow end region, a ratio of area of a given cell to a neighboring cell is 0.25 to 4, a ratio of height of the given cell to the neighboring cell is 0.5 to 2, or an aspect ratio of all cells of the inflow end region is less than 1.6, and wherein in the outflow end region, aspect ratios of a first cell and a second cell in a single circumferential row vary with respect to one another from 1 to 3, areas of the first cell and the second cell vary with respect to one another from 5 mm² to 250 mm², or heights of the first cell and the second cell vary with respect to one another from 3 mm to 30 mm.

12. The replacement heart valve prosthesis of claim 11, wherein the stent component includes at least one feeler for aligning the stent component relative to a native heart valve.

13. The replacement heart valve prosthesis of claim 12, wherein the at least one feeler is configured to be introduced into a native cusp of the native heart valve.

14. The replacement heart valve prosthesis of claim 12, wherein the stent component includes three feelers for aligning the stent component relative to native cusps of the native heart valve.

15. The replacement heart valve prosthesis of claim 12, wherein the stent component includes exactly three feelers for aligning the stent component relative to the native heart valve.

16. The replacement heart valve prosthesis of claim 11, wherein the valve component comprises tissue, and the stent component further comprises an arch for fixation of the tissue.

17. The replacement heart valve prosthesis of claim 16, wherein the tissue comprises pericardial tissue.

18. The replacement heart valve prosthesis of claim 16, wherein the arch comprises holes, rails, or notches.

19. The replacement heart valve prosthesis of claim 11, wherein the stent component comprises a ring of cells at the inflow end region.

20. The replacement heart valve prosthesis of claim 11, wherein the stent component is self-expanding.
